(19) United States
(12) Patent Application Publication
(10) Pub. No.: US 2018/0104106 A1
(43) Pub. Date: Apr. 19, 2018

(54) A SYSTEM AND METHOD FOR DISPLAYING A VIDEO IMAGE

(71) Applicants: AGENCY FOR SCIENCE, TECHNOLOGY AND RESEARCH, Singapore (SG); Tan Tock Seng Hospital Pte Ltd, Singapore (SG)

(72) Inventors: Beng Hai LEE, Singapore (SG); Jiang LIU, Singapore (SG); Wing Kee Damon WONG, Singapore (SG); Ai Ping YOW, Singapore (SG); Tock Han LIM, Singapore (SG); Augustinus LAUDE, Singapore (SG)

(21) Appl. No.: 15/573,447

(22) PCT Filed: May 12, 2016
(86) PCT No.: PCT/SG2016/050222

§ 371 Date: Nov. 10, 2017

(30) Foreign Application Priority Data
May 12, 2015 (SG) 10201503733W

(51) Int. Cl. A61F 9/08; G06T 19/00; G06T 5/00; H04N 5/232; H04N 5/225; G06T 1/00

(52) U.S. Cl. CPC A61F 9/08; G06T 19/006; G06T 1/0007; H04N 5/23248; H04N 5/2259; G06T 5/003

(57) ABSTRACT

The present invention relates to a system for displaying a video image to a user having a visual impairment. The system comprises a video camera, a data storage device, a processor and an image display device for displaying processed images in a display region. The processor is operative to perform at least one of the operations of (i) including in the display region, a marker at a location offset from a centre of the display region, whereby if the user, who suffers from blurred vision at the centre of the user's visual field, looks at the marker, the user sees the captured image in focus; or (ii) deforming a portion of the captured image by a correction matrix, whereby, upon displaying the processed images by the display device, the correction matrix corrects a deformation in a corresponding portion of the visual field of the user due to the visual impairment.

### FIELD OF THE INVENTION

The present invention relates to a system and method for displaying a video image to a user having a visual impairment.

### BACKGROUND OF THE INVENTION

Age-related Macula Degeneration (AMD) is a leading cause of vision loss among older people. In particular, AMD is a common degenerative condition of aging that causes damage to the macula and affects central vision, resulting in low vision. Patients with AMD usually have symptoms including blurred vision or distortion (for example, straight lines appearing wavy and objects appearing to be of an unusual size or shape). Many patients may also develop a scotoma at the fovea. Therefore, patients with AMD tend to face difficulties with simple daily activities such as reading, facial recognition, etc. In addition, to the patients, objects may not appear to be as bright as they used to be.

FIGS. 1(i)-(iv) illustrate how an image appears to a patient as AMD progresses. In particular, FIG. 1(i) illustrates how the image appears to a person with normal vision. FIG. 1(ii) illustrates how the image appears to a patient with early AMD. As shown in FIG. 1(ii), the image appears to contain blurred areas and slight distortion. FIG. 1(iii) illustrates how the image appears to the patient as his/her AMD worsens. In this case, the blurred area becomes larger. FIG. 1(iv) illustrates how the image appears to the patient as his/her AMD worsens further. As shown in FIG. 1(iv), the image appears to contain a black spot.

To assist a patient with a scotoma to read, the location of the scotoma may be defined and the patient may be trained to use a Preferred Retinal Locus (PRL) instead of his/her fovea for fixation on an object of interest. Typically, this involves proper training of the patient and the use of Amsler Grid (which is a diagnostic tool generally used by optometrists to locate and characterize scotomas, and to determine suitable PRLs for patients). FIG. 2(i) shows how an Amsler Grid appears to a person with normal vision and FIG. 2(ii) shows how the Amsler Grid appears to a person with AMD.

In particular, after determining a suitable PRL for a patient, the patient may be trained to use this PRL for fixation on the object of interest. This involves training the patient to move his/her scotoma away from the object of interest. Over time and with proper training, the patient can adapt and develop his/her PRL (which may be different from the determined PRL) at an eccentric (offset) area away from the fovea. This PRL may be described as a “pseudo-fovea” and such a technique may be known as eccentric viewing. In UK, there is even a community-based training program (Macular Society) where skilled volunteers are trained to teach patients eccentric viewing and steady eye strategies.

The teaching and training of the eccentric viewing technique are often challenging. A substantial period of time is required to train AMD patients with scotomas to use their PRLs to look at objects and optometrists have to be around during the training. Furthermore, at present, rehabilitation is not well established and in some cases, the teaching approach has to be individualized.

As an alternative or further to the above-mentioned training, patients having AMD can utilize an appropriate low vision aid to enhance their vision. In fact, many learners of eccentric viewing tend to further require low vision aids due to the impairment of their fovea areas. FIGS. 3(i)-(iii) show examples of such low vision aids. In particular, FIG. 3(i) shows a spectacle-mounted aspheric hyperocular low vision aid. Such a low vision aid usually has a magnification ranging from two times onwards and may be used to enhance the vision of a patient. The optometrist will generally assess the level of magnification to be used via a low-vision assessment of the patient prior to his/her training. Alternative devices to enhance reading also include bright field magnifiers, stand magnifiers and portable video magnifiers (an example of which is shown in FIG. 3(iii)). Intraocular lens implants (e.g. IOL-Vip by Soleko as shown in FIG. 3(ii) and CentraSight by VisionCare Ophathalmic Technologies) have also been developed to meet the needs of patients with low central visions.

Although there are a number of spectacle-mounted low vision aids available in the market, the magnification factors of their magnifying lens are usually fixed. In such cases, a patient can only change the magnification factor by buying a new hyperocular low vision aid to attach to the spectacles. Similarly, the magnification levels for intraocular lens are usually fixed and cannot be changed without a surgical operation to replace the originally implanted lens with one of higher or lower magnification. Moreover, the use of low vision aids or intraocular lens having higher magnification usually results in a smaller field of view and increased distortion for the patient. The shorter working distance associated with the higher magnification can also compromise the amount of light reaching the reading material, making reading difficult and uncomfortable for the patient.

### SUMMARY OF THE INVENTION

The present invention aims to provide a new and useful system and method for displaying a video image to a user having a visual impairment.

In general terms, the present invention proposes a system or method having at least one of the following: including a marker in a display region so as to guide a user to look at the marker to see the image in focus and deforming a portion of the image to correct a deformation in a corresponding portion of the visual field of the user due to the visual impairment.

Specifically, a first aspect of the present invention is a system for displaying a video image to a user having a visual impairment, the system comprising:
a video camera for capturing images;
a data storage device for storing data characterizing the visual impairment;
a processor for processing the captured images in real time in dependence on the data, to generate processed images; and
an image display device for displaying the processed images in real time in a display region for viewing by the user;
wherein the processor is operative to perform at least one of the operations of:
(i) including in the display region a marker at a location determined by said data offset from a centre of the display region, whereby if the user, who suffers from blurred vision at the centre of the user's visual field, looks at the marker, the user sees the captured image in focus; or
(ii) deforming a portion of the captured image which depends on the data, by a correction matrix which depends upon the data, whereby, upon displaying the processed images by the display device, the correction matrix corrects a deformation in a corresponding portion of the visual field of the user due to the visual impairment.

With the above-mentioned operation (i), the system is able to use the marker to guide the user to use his/her PRL to view the captured image. This can allow the user to more easily view the captured image with his/her PRL as this requires less training and can be done in the absence of an optometrist. With the above-mentioned operation (ii), the system is able to allow the user to see the captured image with less distortion, therefore allowing the user to feel that his/her vision has been enhanced.

A second aspect of the present invention is a method for displaying a video image to a user having a visual impairment, the method comprising:
capturing images with a video camera;
processing the captured images in real time in dependence on data characterizing the visual impairment, to generate processed images; and
displaying the processed images to the user in real time in a display region;
wherein the processing includes at least one of the operations of:
(i) including in the display region a marker at a location determined by said data offset from a centre of the display region, whereby when the processed images are displayed to the user, who suffers from blurred vision at the centre of the user's visual field, and the user looks at the marker, the user sees the captured image in focus; or
(ii) deforming a portion of the captured image which depends on the data, by a correction matrix which depends upon the data, whereby the correction matrix corrects a deformation in a corresponding portion of the visual field of the user due to the visual impairment.

A third aspect of the present invention is an image processing module for processing images captured with a video camera in real time to generate processed images for display in real time, in a display region, to a user having a visual impairment, wherein the processing is in dependence on data characterizing the visual impairment and wherein the image processing module comprises:
an image receiving module configured to receive the captured images;
a data receiving module configured to receive the data;
and one or both of:
(i) an image focusing module configured to include in the display region, a marker at a location determined by said data offset from a centre of the display region, whereby if the user, who suffers from blurred vision at the centre of the user's visual field, looks at the marker, the user sees the captured image in focus;
(ii) an image correction module configured to deform a portion of the captured image which depends on the data, by a correction matrix which depends upon the data, whereby the correction matrix corrects a deformation in a corresponding portion of the visual field of the user due to the visual impairment.

The processor of the first aspect may comprise the image processing module of the third aspect.

### BRIEF DESCRIPTION OF THE FIGURES

An embodiment of the invention will now be illustrated for the sake of example only with reference to the following drawings, in which:

FIGS. 1(i)-(iv) show how an image appears to a patient as AMD progresses;

FIGS. 2(i)-(ii) respectively show how an Amsler Grid appears to a person with normal vision and a person with AMD;

FIGS. 3(i)-(iii) show examples of low vision aids;

FIG. 4 shows a system for displaying a video image to a user having a visual impairment according to an embodiment of the present invention;

FIG. 5 shows a flow diagram of a method performed by the system of FIG. 4;

FIG. 6 shows the operations the system of FIG. 4 can perform;

FIG. 7 shows sub-steps of a step of the method of FIG. 5 for generating data characterizing a user's visual impairment in the form of eccentric fixation;

FIG. 8 shows a screen presented to a user during a sub-step of FIG. 7;

FIG. 9 shows sub-steps of a step of the method of FIG. 5 for processing an image to generate a processed image using the data from the sub-steps of FIG. 7;

FIGS. 10(i)-(ii) show an example implementation of the sub-steps of FIG. 9;

FIG. 11 shows sub-steps of a step of the method of FIG. 5 for generating data characterizing a user's visual impairment in the form of visual field distortion;

FIG. 12 shows sub-steps of a step of the method of FIG. 5 for processing an image to generate a processed image using the data from the sub-steps of FIG. 11;

FIGS. 13(i)-(iii) show an example implementation of the sub-steps of FIG. 12;

FIG. 14 shows sub-steps of a step of the method of FIG. 5 for magnifying an image by a selectable magnification factor to generate a processed image;

FIGS. 15(i)-(ii) show an example implementation of the sub-steps of FIG. 14;

FIGS. 16(i)-(iii) show another example implementation of the sub-steps of FIG. 14;

FIG. 17 shows sub-steps of a step of the method of FIG. 5 for generating data characterizing a user's visual impairment in the form of a decreased ability to view contrast in images;

FIG. 18 shows sub-steps of a step of the method of FIG. 5 for processing an image to generate a processed image using the data from the sub-steps of FIG. 17;

FIGS. 19(i)-(ii) show an example implementation of the sub-steps of FIG. 18;

FIGS. 20(i)-(ii) respectively show consecutive video image frames as seen by a stationary user and a non-stationary user; and

FIG. 21 shows sub-steps of a step of the method of FIG. 5 for image stabilization.

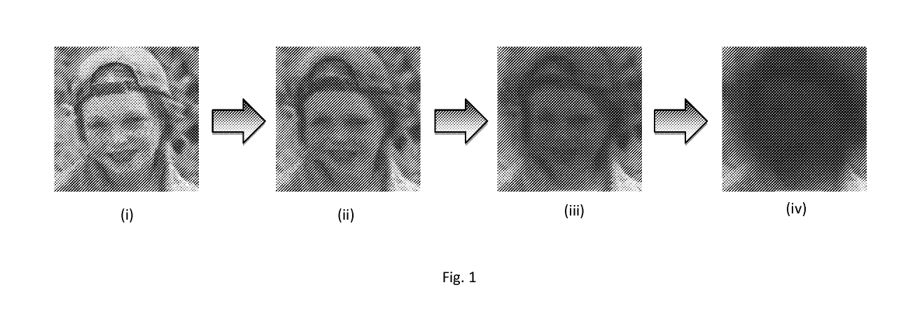

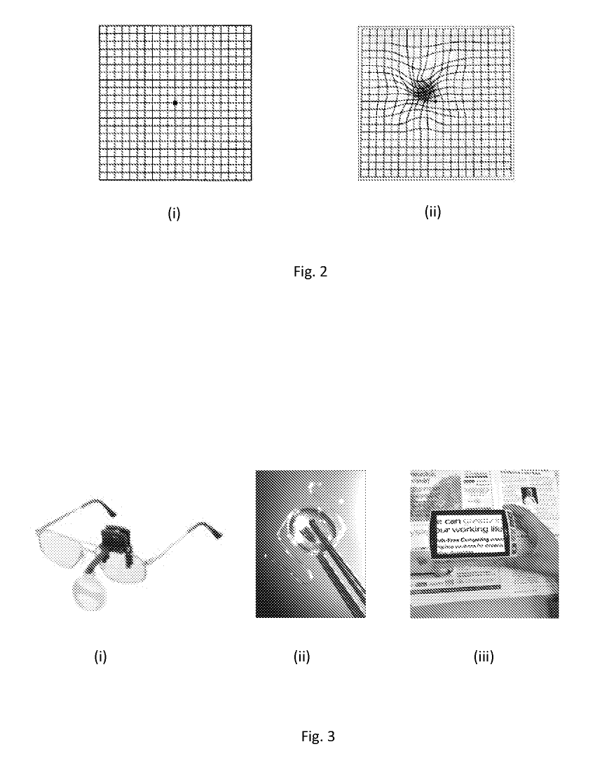

### DETAILED DESCRIPTION OF THE EMBODIMENTS

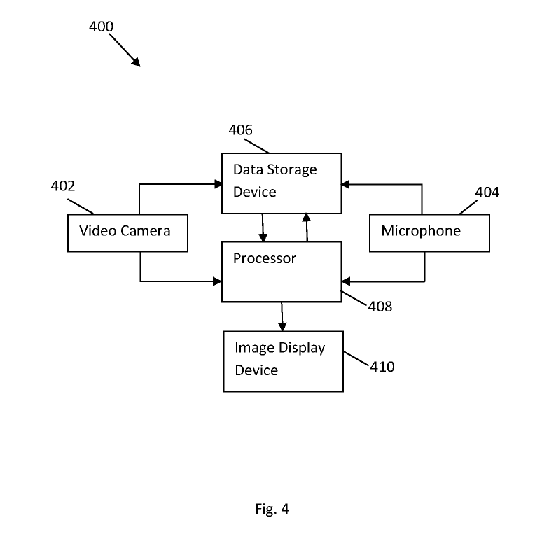

FIG. 4 shows a system 400 for displaying a video image to a user having a visual impairment according to an embodiment of the present invention. The system 400 may also be known as an Automated Low Vision Aid (ALVA).

The system 400 may be adapted for wearing by a user, for example, by further comprising elements operative to attach the system 400 to the user. For example, the system 400 may be in the form of a wearable device such as goggles which can be worn by the user daily.

Alternatively, the system 400 may be in the form of a small handheld device such as a smart phone, a tablet or a phablet that a user can hold and place in front of his/her eyes.

As shown in FIG. 4, the system 400 comprises a video camera 402, a microphone 404, a data storage device 406, a processor 408 and an image display device 410. The video camera 402 may be part of an eye gear worn by the user.

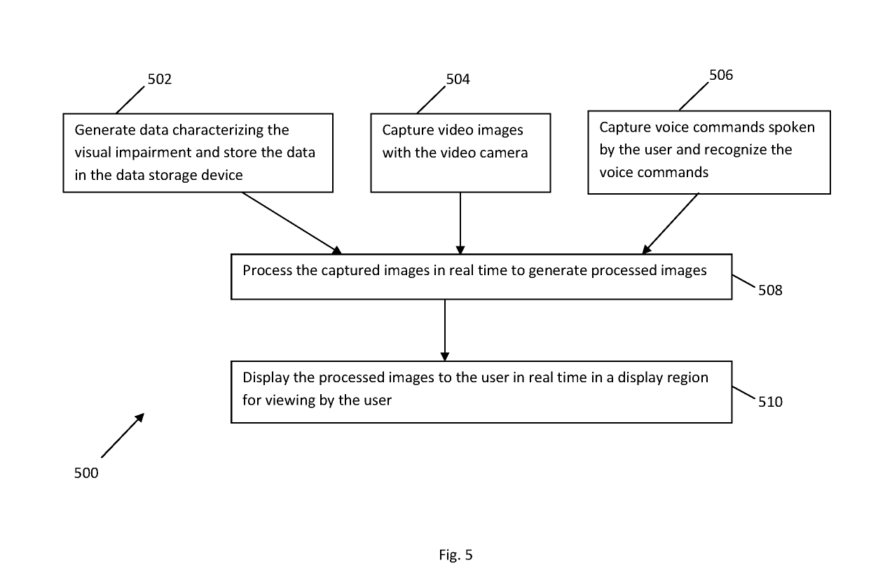

FIG. 5 shows a flow diagram of a method 500 performed by the system 400 for displaying a video image to a user having a visual impairment according to an embodiment of the present invention. The method 500 comprises steps 502-510.

In this document, a video image is used to refer to an image frame captured by the video camera 402 (either most recently captured or has been processed by one or more image enhancement operations). This video image is 2-dimensional and comprises a plurality of pixels having respective x and y coordinates, and respective intensity values.

In step 502, data characterizing the visual impairment is generated by measuring characteristics of the visual impairment and is stored in the data storage device 406. In step 504, images are captured with the video camera 402. In step 506, voice commands spoken by the user are captured using the microphone 404 and are recognized by the processor 408. In step 508, the captured images are processed in real time by the processor 408 to generate processed images. This processing is dependent on the data characterizing the visual impairment generated in step 502. The processing may be modified based on the recognized voice commands captured in step 506. In step 510, using the image display device 410, the processed images are displayed to the user in real time in a display region for viewing by the user. The display region is configured to display a plurality of pixels in 2-dimension.

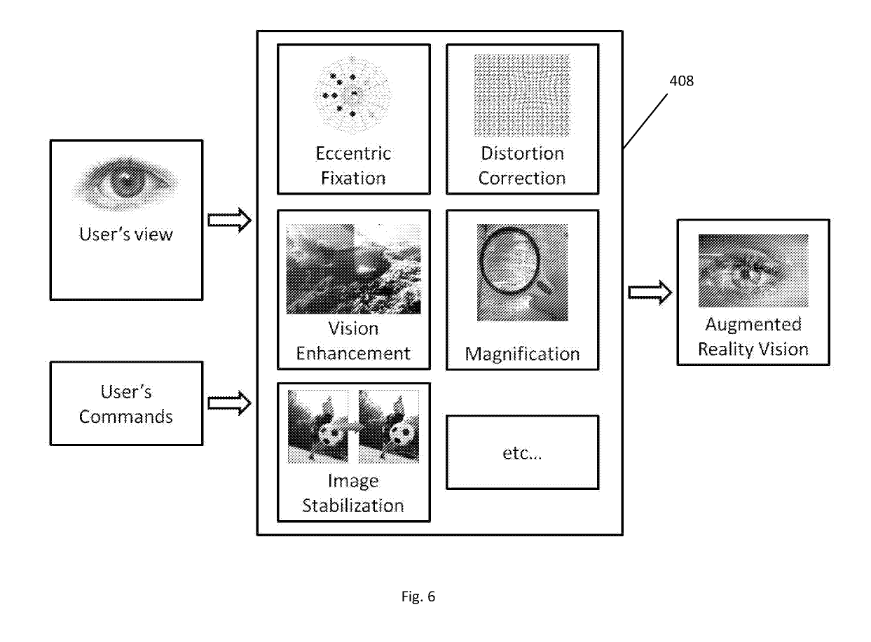

The processor 408 of system 400 is configured such that it is able to perform multiple image enhancement operations including eccentric fixation, distortion correction, vision enhancement, magnification and image stabilization. These are shown in FIG. 6. In particular, as shown in FIG. 6, the input to processor 408 comprises the user's view in the form of images captured by the video camera 402. These images comprise real time video images (i.e. image frames of a video) as seen from the user's perspective. The input to processor 408 also comprises user's commands captured using the microphone 404. The processor 408 is configured to perform one or more of the above-mentioned operations on the captured images to generate processed images. These processed images are enhanced versions of the captured images and provide the user augmented reality vision.

### Eccentric Fixation

A patient suffering from eccentric fixation tends to suffer from blurred vision at the center of his/her visual field, causing him/her to see the center of an image as a blurred area. The eccentric fixation operation allows the patient to use his/her PRL to view an object of interest, so that the object of interest can appear clear to him/her.

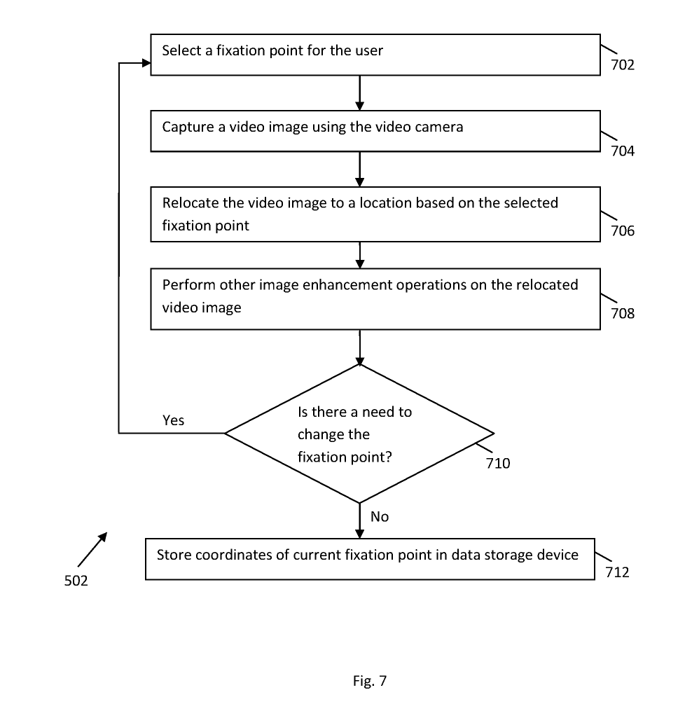

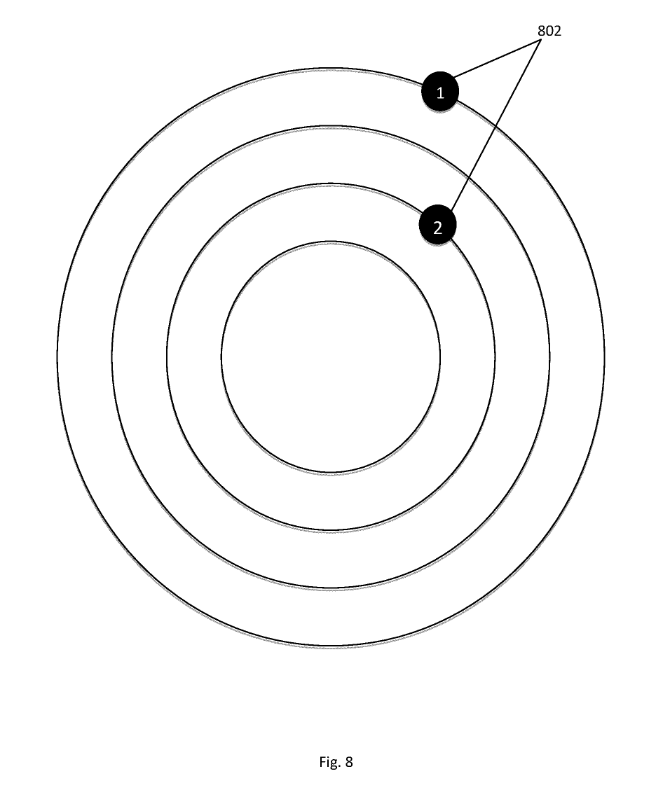

FIG. 7 shows sub-steps 702-712 of step 502 for generating data characterizing a user's visual impairment in the form of eccentric fixation.

In sub-step 702, a fixation point for the user is selected. The fixation point has coordinates [xf, yf] where xf is less than the total number of pixels that the display region of the image display device 410 can display along the x-axis and yf is less than the total number of pixels the display region can display along the y-axis. In particular, the fixation point is selected by presenting a screen to the user on the display region for the user to select a point on the screen. The point selected by the user is then set as the user's fixation point. The screen used in sub-step 702 may comprise markers to guide the user. In this case, the user may be asked to fix his/her glance at each marker for a certain period of time and then select the marker he/she can see clearly. The selected marker is then set as the user's fixation point.

FIG. 8 shows an example of a screen that can be used in sub-step 702. As shown in FIG. 8, the screen comprises a plurality of concentric circles with the centre of the innermost circle corresponding to the user's default retinal locus. Markers 802 are further included on circles of different radii. As mentioned above, the user may be asked to select the marker he/she can see clearly and this selected marker is then set as the user's fixation point. The screen may alternatively comprise the Amsler Grid (but this is not necessary).

In sub-step 704, a video image is captured using the video camera 402.

In sub-step 706, the video image is relocated to a location based on the selected fixation point.

In particular, sub-step 706 comprises translating the video image to a location such that the center of the image is at the selected fixation point of the display region.

In sub-step 708, other image enhancement operations (as shown in FIG. 6) as desired by the user are performed on the relocated image to obtain a processed image.

In sub-step 710, it is determined whether there is a need to change the fixation point. In particular, a marker is included in the display region. The location of this marker corresponds to the location of the fixation point which is offset from a centre of the display region. The user is then asked to look at the marker. At this marker, the user is able to see the processed image. The user is then asked to indicate whether the processed image he/she sees is in focus. If the user indicates that the processed image is not clear, it is determined that there is a need to change the fixation point and sub-steps 702-710 are repeated with a new fixation point selected in sub-step 702. This new fixation point is selected by shifting the current fixation point by a predetermined number of pixels in the x and/or y direction. Otherwise, if the user indicates that the processed image is clear, it is determined that there is no need to change the fixation point and sub-step 712 is performed in which the [xf, yf] coordinates of the current fixation point are stored in the data storage device 406.

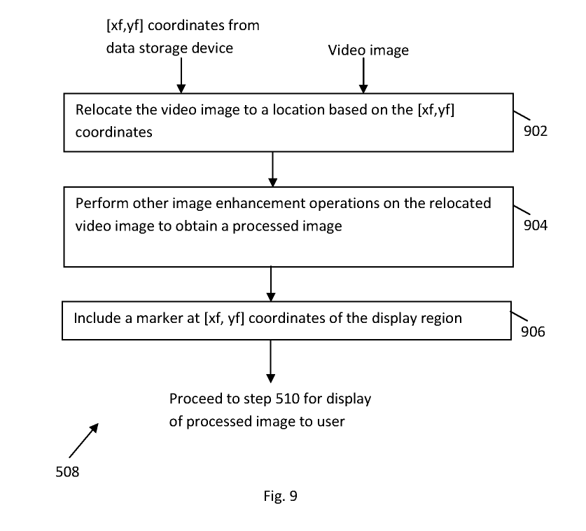

FIG. 9 shows sub-steps 902-906 of step 508 for processing an image to generate a processed image using the data (more specifically, [xf, yf] coordinates) generated from sub-steps 702-712 and stored in the data storage device 406. These sub-steps 902-906 are for the eccentric fixation operation shown in FIG. 6.

Besides the [xf, yf] coordinates from the data storage device 406, the input to sub-steps 902-906 further comprises a video image which may be one that is most recently captured using the video camera 402 or one that has been processed by one or more operations of the system 400 as shown in FIG. 6.

In sub-step 902, the input video image is relocated to a location based on the [xf, yf] coordinates of the fixation point. Specifically, the video image is translated to a location such that the center of the video image is at the [xf, yf] coordinates of the display region.

In sub-step 904, other image enhancement operations (as shown in FIG. 6) as desired by the user are performed on the relocated image to obtain a processed image.

In sub-step 906, a marker is included at the [xf,yf] coordinates of the display region.

The processed image is then displayed to the user at the [xf, yf] coordinates of the display region in step 510 of method 500. The [xf, yf] coordinates are offset from a centre of the display region as the user is one who suffers from blurred vision at the centre of his/her visual field. These coordinates are indicative of the user's PRL since they are obtained using sub-steps 702-712. Accordingly, by displaying the processed image with its centre at the [xf, yf] coordinates of the display region, if the user looks at the marker, the user sees the captured image in focus.

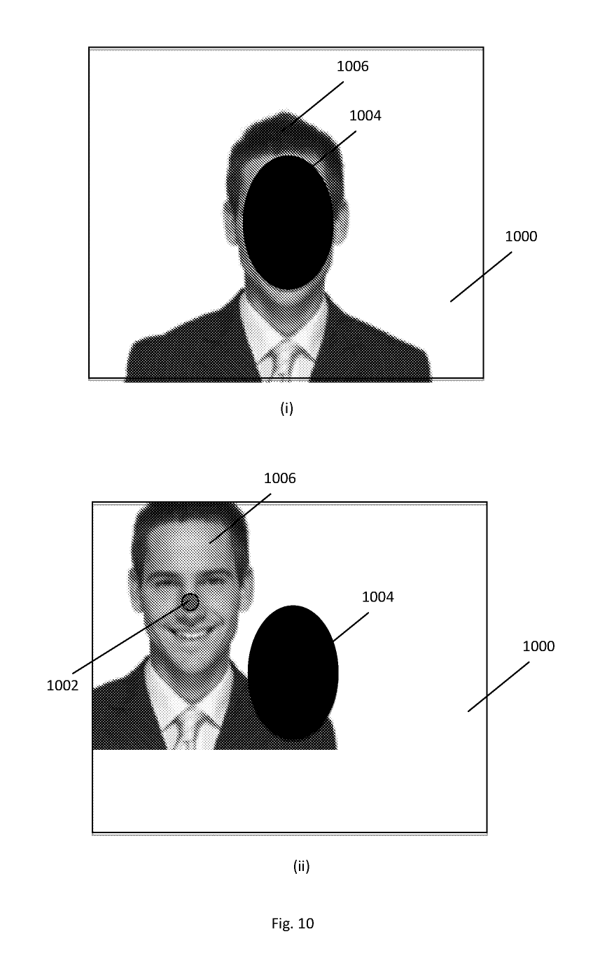

FIGS. 10(i)-(ii) show an example implementation of sub-steps 902-906. In particular, FIGS. 10(i) and (ii) respectively show the display region 1000 as seen from the user's perspective before and after performing sub-steps 902-906. As shown in FIG. 10(i), due to the user's scotoma 1004, the user has difficulty viewing the object of interest 1006 in the captured image. In particular, the user is unable to see the face of the object of interest 1006. After performing sub-steps 902-906, the image is relocated to a location, such that the centre of the image is at the [xf, yf] coordinates offset from a centre of the display region 1000. A marker 1002 is further included in the display region 1000 at the [xf, yf] coordinates. This is shown in FIG. 10(ii). The user can thus see the captured image in focus in FIG. 10(ii) by looking at the marker 1002.

### Distortion Correction

A patient suffering from visual field distortion tends to see a portion of his/her visual field as deformed. As a result, when looking at an image, the patient sees the portion of the image corresponding to the deformed portion of his/her visual field as distorted or deformed. This portion of the image may be termed as the user's zone of distortion in the image.

FIG. 11 shows sub-steps 1102-1114 of step 502 for generating data (in particular, zone of distortion points' coordinates) characterizing a user's visual impairment in the form of visual field distortion.

In sub-step 1102, a video image is captured using the video camera 402.

In sub-step 1104, a user distortion matrix characterizing an image deformation caused to at least a portion of the visual field of the user by the visual impairment is defined. This user distortion matrix is defined based on the user's zone of distortion in the captured image as input by the user. Specifically, the user is shown the captured video image with a grid overlaid on the image and the user is requested to input to system 400 the coordinates of two points defining his/her zone of distortion on the grid, specifically, point_top_left (the point corresponding to the top left hand corner of his/her zone of distortion) and point_bottom_right (the point corresponding to the bottom right hand corner of his/her zone of distortion). This may be done by having the user input the coordinates of these two points using verbal commands via the microphone or by displaying the video image on a touch screen and requesting the user to touch the above-mentioned points. Using the coordinates of the above-mentioned two points, the system 400 automatically marks out the user's zone of distortion by first setting (i) all the pixels of the image having the same x coordinates as point_top_left, (ii) all the pixels having the same y coordinates as point_top_left, (iii) all the pixels having the same x coordinates as point_bottom_right and (iv) all the pixels having the same y coordinates as point_bottom_right as the boundary of the zone of distortion. Next, the zone of distortion is marked by setting this zone as comprising all the pixels within the boundary and all the pixels of this boundary. The user distortion matrix M is then defined by setting the user distortion matrix M as a 2-dimensional matrix having entries respectively corresponding to the pixels of the marked zone of distortion, with the values of these entries being the intensity values of the respective pixels.

In sub-step 1106, a correction matrix M′ is generated by inverting the user distortion matrix M. The purpose of this correction matrix M′ is to correct the user distortion matrix M.

In sub-step 1108, the correction matrix M′ is applied to the video image captured in sub-step 1102 to obtain a deformed image. More specifically, applying the correction matrix M′ to the video image deforms a portion of the image (this portion corresponds to the zone of distortion input by the user).

In sub-step 1110, other image enhancement operations (as shown in FIG. 6) as desired by the user are performed on the deformed image to obtain a processed image.

In sub-step 1112, it is determined whether there is a need to redefine the correction matrix M′. In particular, the user is asked if the processed image is now clear and if not, it is determined that there is a need to redefine the correction matrix M′ and sub-steps 1102-1112 are repeated. Else, if the user finds the processed image clear, it is determined that there is no need to redefine the correction matrix M′ and sub-step 1114 is performed in which the coordinates of the current points defining the user's zone of distortion i.e. point_top_left and point_bottom_right are stored as zone of distortion points' coordinates in the data storage device 406.

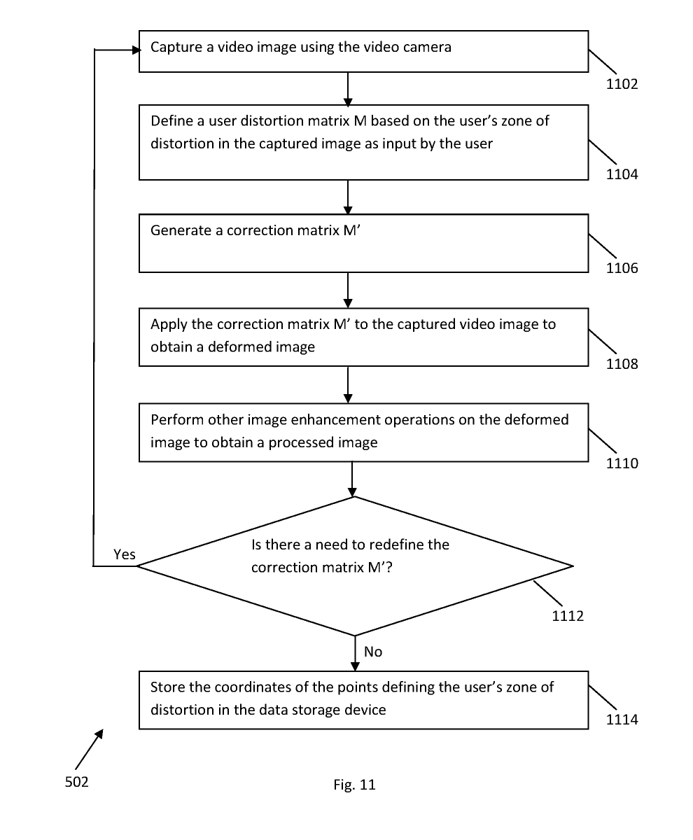

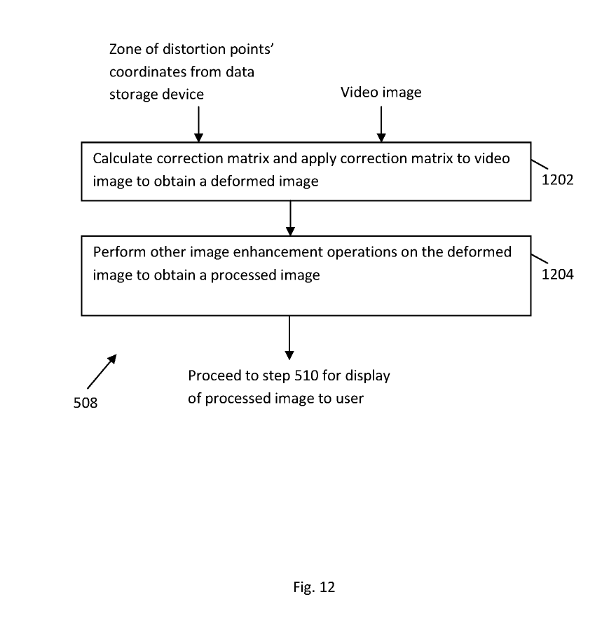

FIG. 12 shows sub-steps 1202-1204 of step 508 for processing an image to generate a processed image using the data (more specifically, the zone of distortion points' coordinates generated from sub-steps 1102-1114 and stored in the data storage device 406). Sub-steps 1202-1204 are for the distortion correction operation shown in FIG. 6.

Besides the zone of distortion points' coordinates from the data storage device 406, the input to sub-steps 1202-1204 further comprises a video image which may be one that is most recently captured using the video camera 402 or one that has been processed by one or more operations of the system 400 as shown in FIG. 6.

In sub-step 1202, a correction matrix M′ is calculated and then applied to the input video image to obtain a deformed image. In particular, using the zone of distortion points' coordinates, the system 400 automatically marks out the user's zone of distortion by first setting (i) all the pixels of the image having the same x coordinates as point_top_left, (ii) all the pixels having the same y coordinates as point_top_left, (iii) all the pixels having the same x coordinates as point_bottom_right and (iv) all the pixels having the same y coordinates as point_bottom_right as the boundary of the zone of distortion. Next, the zone of distortion is marked by setting this zone as comprising all the pixels within the boundary and all the pixels of this boundary. A user distortion matrix M is then defined by setting the user distortion matrix M as a 2-dimensional matrix having entries respectively corresponding to the pixels of the marked zone of distortion, with the values of these entries being the intensity values of the respective pixels. The correction matrix M′ is then calculated as the inverse of the user distortion matrix M and applied to the image. This causes a portion of the image to be deformed (or in other words, augmented) by the correction matrix M′. This portion of the image depends on the above-mentioned zone of distortion marked out by the system 400 using the zone of distortion points' coordinates obtained from sub-steps 1102-1114.

Other image enhancement operations (as shown in FIG. 6) as desired by the user are then performed on the deformed image in sub-step 1204 to obtain a processed image and the processed image is then displayed to the user in step 510.

Specifically, the portion of the image to be deformed in sub-step 1202 corresponds to the zone of distortion input by the user (in sub-steps 1102-1114), and the deformation corrects the portion so that the image appears undistorted to the user (whereas the deformed image is likely to appear distorted to a person with normal vision). In other words, upon displaying the processed images by the display device 410, the correction matrix corrects the deformation in the corresponding portion of the visual field of the user due to the visual impairment. For example, a user with visual impairment may see a straight line as a curve. With the application of the correction matrix to the image comprising the straight line, the straight line can appear closer to its original form (i.e. straighter) to the user.

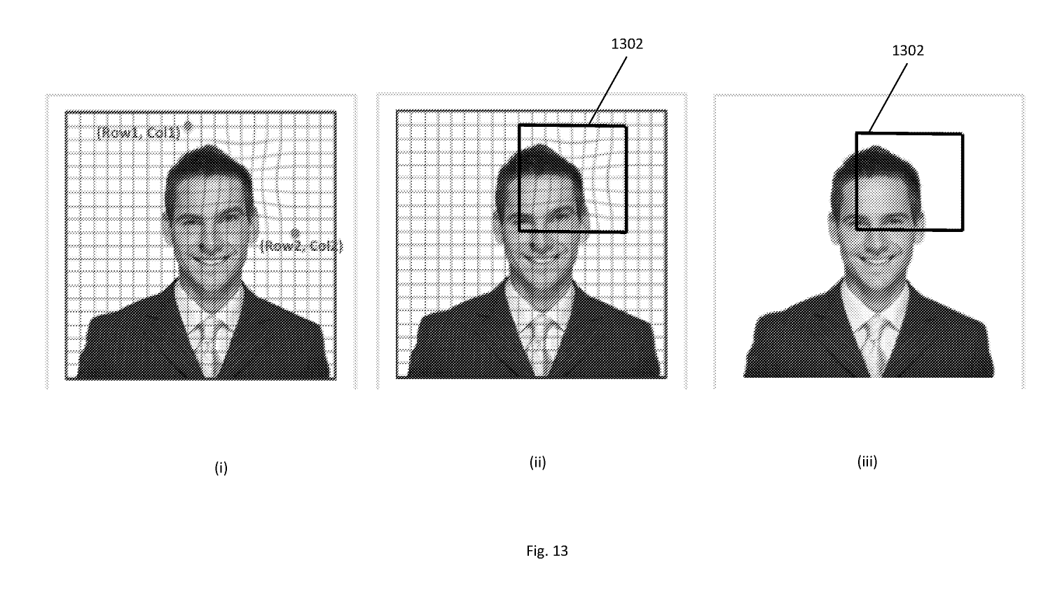

FIGS. 13(i)-(iii) shows an example implementation of sub-steps 1202-1204. In particular, FIG. 13(i) shows a captured image as seen from the user's perspective, with a grid overlaid on the image. FIG. 13(i) further shows the coordinates (Row1, Col1) and (Row2, Co12) which are the zone of distortion points' coordinates in this example. Specifically, (Row1, Col1) are the coordinates of the point corresponding to the top left hand corner of the user's zone of distortion and (Row2, Co12) are the coordinates of the point corresponding to the bottom right hand of the user's zone of distortion. FIG. 13(ii) shows the boundary 1302 of the zone of distortion marked out by system 400 and FIG. 13(iii) shows the zone of distortion within and including the boundary 1302 corrected by the correction matrix as seen from the user's perspective.

### Magnification

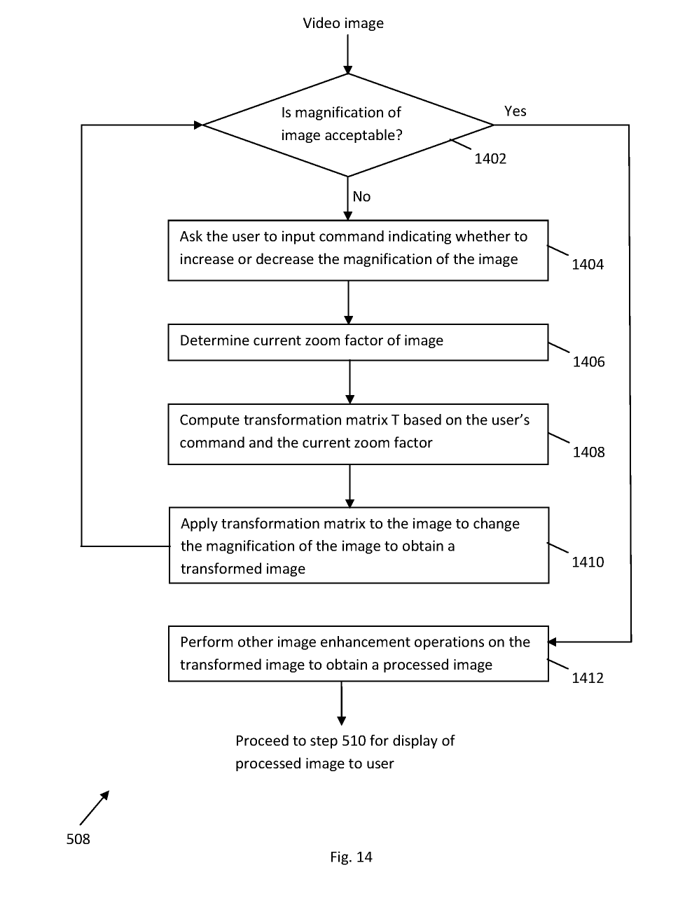

FIG. 14 shows sub-steps 1402-1412 of step 508 for processing an image to generate a processed image whereby the processing comprises magnifying the image by a selectable magnification factor. In other words, sub-steps 1402-1412 are for the magnification operation shown in FIG. 6.

The input to sub-steps 1402-1412 comprises a video image which may be one that is most recently captured using the video camera 402 or one that has been processed by one or more operations of the system 400 as shown in FIG. 6.

In sub-step 1402, the user is asked if the magnification of the image is acceptable and if so, sub-step 1412 is performed in which other image enhancement operations (as shown in FIG. 6) as desired by the user are performed to obtain a processed image and the processed image is then displayed to the user. If the user finds the magnification of the image not acceptable, sub-steps 1404-1410 are performed.

In sub-step 1404, the user is asked to input a command (which may be a verbal command via the microphone 404) indicating whether the user wishes to increase or decrease the magnification of the image.

The current zoom factor of the image is then determined in sub-step 1406. In particular, the zoom factor is stored in the data storage device 406. The zoom factor has a default value (e.g. 1) when the system 400 starts operation and each time the user indicates that he/she wishes to increase/decrease the magnification of the image, the zoom factor is increased/decreased by a certain amount (e.g. 1) and stored in the data storage device 406. For example, if the zoom factor is at the default value 1 and the user indicates his/her wish to increase the magnification of the image, the zoom factor is increased to 2. In sub-step 1406, the current zoom factor is determined by retrieving the zoom factor from the data storage device 406.

In sub-step 1408, a transformation matrix T is computed based on the user's command and the current zoom factor. In sub-step 1410, the transformation matrix T is applied to the image to obtain a transformed image. In particular, the transformation matrix is computed in sub-step 1408 to magnify the image by a particular zoom factor. This particular zoom factor is calculated based on the user's command and the current zoom factor. For example, if the current zoom factor is at its default value 1 and the user indicates his/her wish to magnify the image, the transformation matrix is computed so that after applying the transformation matrix to the image in sub-step 1410, the image is magnified by 2×. Take for example a display region in the form of a bitmap of dimension w×h. In this case, if the current zoom factor is 1 and the user wishes to magnify the image, the bitmap is scaled up to the size of 2w×2h and then cropped to the size of w×h, the scaling and cropping being done while keeping the centroids of both the original bitmap and the scaled up bitmap invariant.

The user is then asked again in sub-step 1402 if the magnification of the image is now acceptable and if not, sub-steps 1404-1410 are repeated. If so, sub-step 1412 is performed.

In sub-step 1412, other image enhancement operations (as shown in FIG. 6) as desired by the user are performed on the transformed image to obtain a processed image. The processed image is then displayed to the user.

Therefore, with system 400, the magnification of the real time video images can be adjusted according to the users' instructions. More specifically, by inputting commands such as verbal commands into system 400, users are able to increase or decrease the level of magnification until they find the transformed image acceptable.

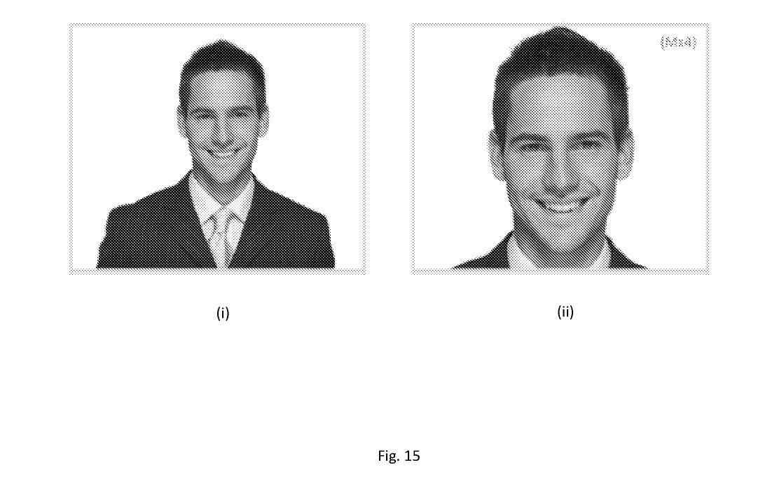

FIGS. 15(i)-(ii) show an example implementation of the sub-steps 1402-1412. In particular, FIG. 15(i) shows an example image and FIG. 15(ii) shows a magnified version of the example image whereby the magnified version is obtained using sub-steps 1402-1412.

Although magnifying an image helps to enlarge an image so the user can see details of the image more clearly, it narrows down the user's field of view causing the user to see less of the image. As a result, the user has to physically move his/her head (if the system 400 is in the form of goggles) or move the handheld apparatus (if the system 400 is in the form of such an apparatus) so as to scan the surroundings until the user can see the objects he/she is interested in. To reduce the amount of scanning the user has to do, the sub-step 1410 may further comprise automated object tracking. In particular, automated object tracking may be performed to identify one or more objects of interest in the image and adjust a location of the image in the display region (after magnifying the image), so that the objects of interest the user are interested in are made visible to the user. For example, an object of interest may be placed with its centre at the centre of the display region. In another example, the object of interest may be placed with its centre at a fixation point (corresponding to the user's PRL) selected in a manner similar to that as described in sub-step 702 above (in this case, the object may be initially placed at the centre of the display region and then translated to the fixation point in sub-step 702 of an eccentric fixation operation performed in sub-step 1412). The automated object tracking may be implemented using object and face detection techniques known in the art.

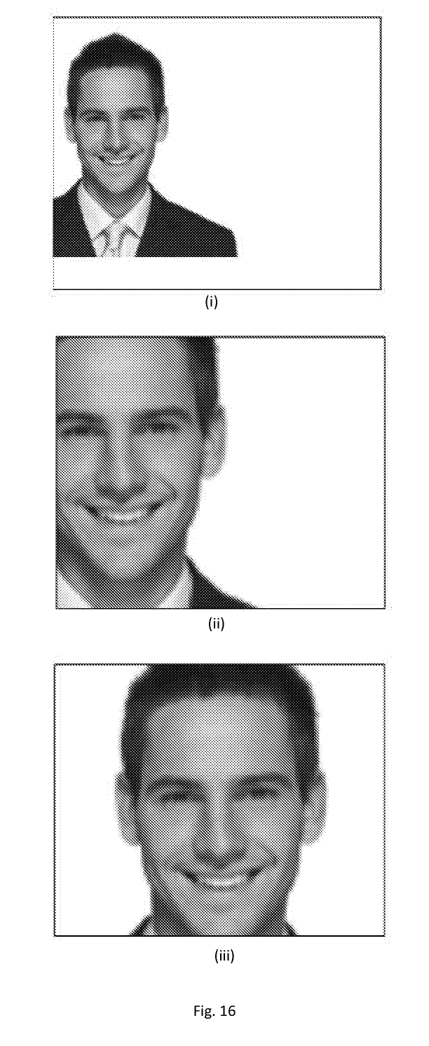

FIGS. 16(i)-(iii) shows the implementation of sub-steps 1402-1412 with automated object tracking performed in sub-step 1410. In particular, FIG. 16(i) shows an example image. FIG. 16(ii) shows the magnification of the example image to 2× its original size and FIG. 16(iii) shows the automated object tracking process which detects the object of interest (specifically, the face) and places the object of interest such that its centre is roughly in the centre of the display region.

### Vision Enhancement

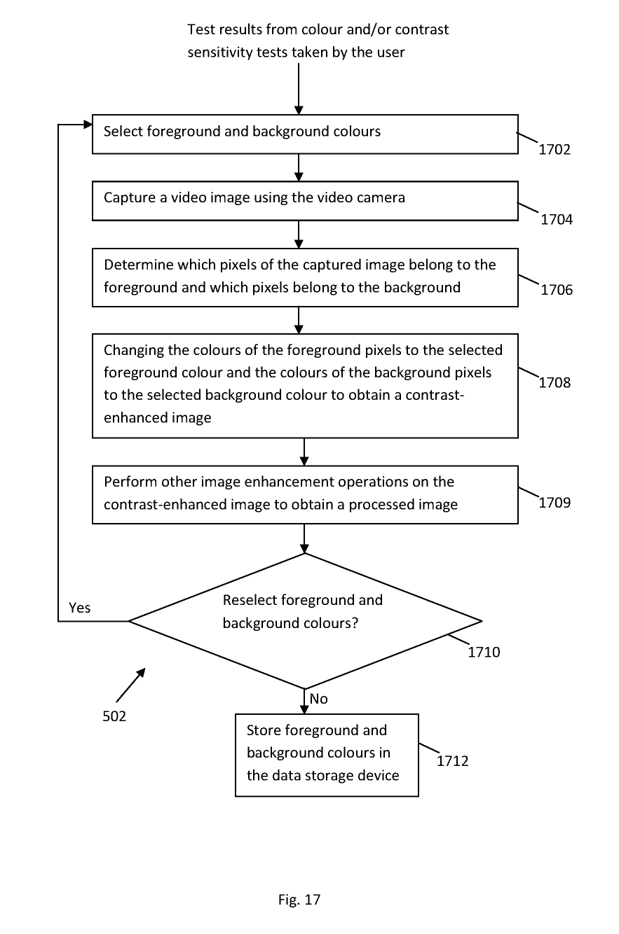

FIG. 17 shows sub-steps 1702-1712 of step 502 for generating data characterizing a user's visual impairment in the form of a decreased ability to view contrast in images (resulting in a decreased ability to read text in images).

Prior to performing sub-steps 1702-1712, the user is asked to take one or more colour and/or contrast sensitivity tests, for example a test based on the colour confusion axis. The results from these tests are then input into sub-steps 1702-1712.

In sub-step 1702, foreground and background colours are selected. The initial foreground and background colours are selected based on the test results of the colour and/or contrast sensitivity test(s) of the user.

In sub-step 1704, a video image comprising text is captured using the video camera 402.

In sub-step 1706, it is determined which pixels of the captured image belong to the foreground (i.e. which are the foreground pixels) and which pixels of the image belong to the background (i.e. which are the background pixels). In particular, the pixels forming the text are determined to belong to the foreground whereas the rest of the pixels are determined to belong to the background.

In sub-step 1708, the colours of the foreground pixels are changed to the selected foreground colour and the colours of the background pixels are changed to the selected background colour to obtain a contrast-enhanced image.

In sub-step 1709, other image enhancement operations (as shown in FIG. 6) as desired by the user are performed on the contrast-enhanced image to obtain a processed image.

In sub-step 1710, it is determined if the foreground and background colours have to be re-selected. In particular, the processed image is displayed to the user and the user is asked to input a command to indicate if he/she finds the processed image acceptable. If the user finds the processed image acceptable, the selected foreground and background colours are stored in the data storage device 406 in sub-step 1712. If not, sub-steps 1702-1710 are repeated with new foreground and background colours selected in sub-step 1702.

The foreground and background colours (both initial and new) can be selected in many ways. For example, a colour palette comprising commonly used colours may be presented to the user for the user to choose the foreground and background colours. In another example, the entire rainbow spectrum of colours may be presented to the user for the user to choose the colours. In yet another example, if the user is particular about the exact shade of his/her desired colours, the user can enter the exact Red, Green and Blue components for the colours he/she wants.

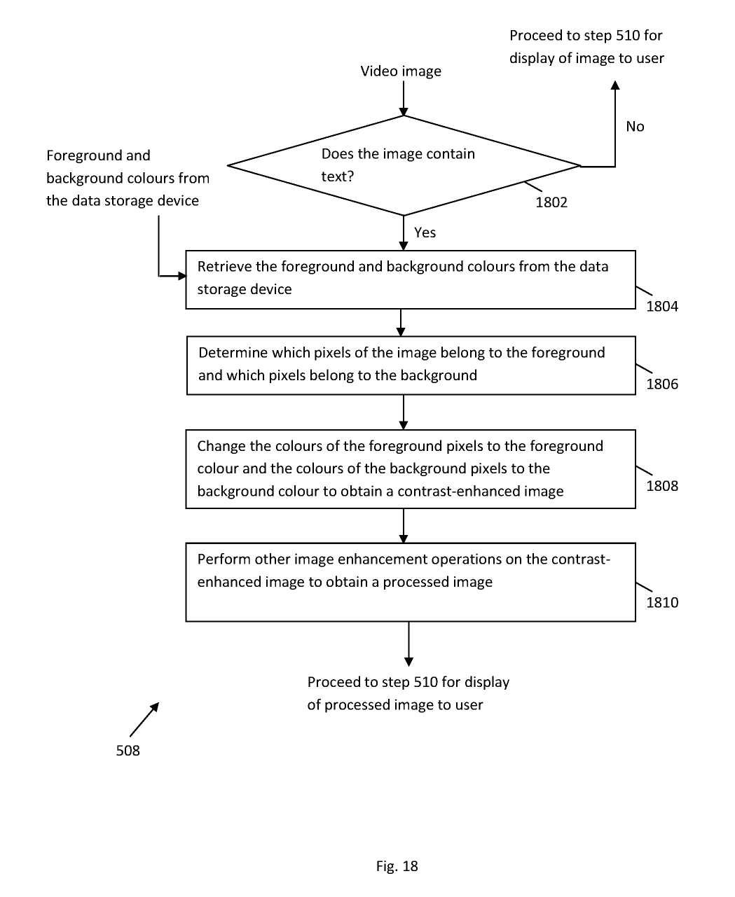

FIG. 18 shows sub-steps 1802-1810 of step 508 for processing an image to generate a processed image using the data (more specifically, the selected foreground and background colours generated from sub-steps 1702-1712 and stored in the data storage device 406). Sub-steps 1802-1810 are for the vision enhancement operation shown in FIG. 6. More specifically, the processing comprises enhancing the images by adjusting the colour, contrast and/or the sharpness of the images. This enhancement can be done based on voice commands provided by the users.

The input to sub-steps 1802-1810 comprises a video image which may be one that is most recently captured using the video camera 402 or one that has been processed by one or more operations of the system 400 as shown in FIG. 6.

In sub-step 1802, it is determined if the input image comprises text. If not, the operation ends, the image is displayed to the user and the next video image frame is input to sub-step 1802. If the input image comprises text, sub-steps 1804-1810 are performed.

In sub-step 1804, the foreground and background colours stored in the data storage device are retrieved.

In sub-step 1806, it is determined which pixels of the image belong to the foreground and which belong to the background. In particular, the pixels forming the text of the image are determined to belong to the foreground and the rest of the pixels are determined to belong to the background.

In sub-step 1808, the colours of the foreground pixels are changed to the foreground colour retrieved from the data storage device and the colours of the background pixels are changed to the background colour retrieved from the data storage device to obtain a contrast-enhanced image.

In sub-step 1810, other image enhancement operations (shown in FIG. 6) as desired by the user are performed on the contrast-enhanced image to obtain a processed image. The processed image is then displayed to the user.

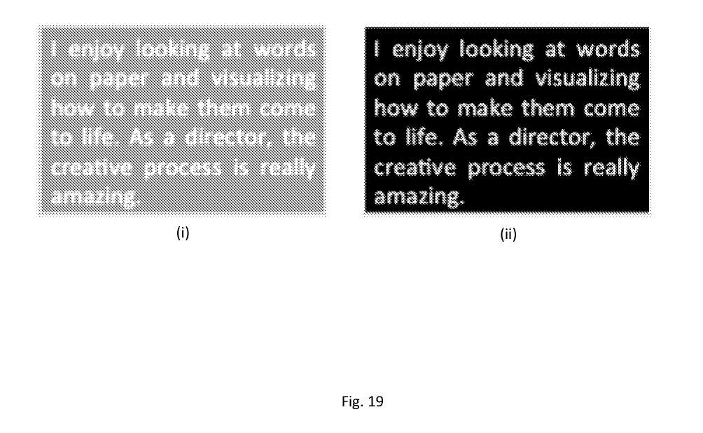

By changing the colour of both the text and background of an image containing text, the contrast between the text and background can be enhanced and users can read the text of the image more easily. FIGS. 19(i)-(ii) shows an example implementation of sub-steps 1802-1810. In particular, FIG. 19(i) shows an initial input image and FIG. 19(ii) shows the processed image after performing sub-steps 1802-1810. As shown in FIG. 19(ii), the text can be read more easily by changing the colours of the text and background of the image.

Besides enhancing the contrast between the text and background of an image, other adjustments of the contrast, sharpness and/or colours of images can be performed. For example, sub-steps similar to those in FIGS. 17 and 18 may be performed on images without text and in this example, the determination of the foreground and background pixels may be based on user input or training data.

Furthermore, in another embodiment, the text in the input image is enhanced using least colour confusion axis and an iterative method. A calibration process is first performed for a particular user to obtain characteristics of the enhancement for the user. These characteristics are saved as part of the user's profile. These saved characteristics are then used to enhance the images with detected text when processing the images in real time. In one example, the calibration process comprises having the user configure multiple reading profiles and loading these profiles when necessary. For example, a red/green colour blind user tends to confuse the colours blue and purple. During the calibration process, such a user can configure his/her settings to indicate that blue and purple colours in images shall be replaced with other colours that appear less confusing to them. The user can start with a particular colour setting and after using this setting for a period of time, the user can change this to a new colour setting and save the new colour setting in his/her profile (either replacing the existing profile or adding to the collection of the user's profiles). The user can repeat this as many times as he/she wishes. In other words, the user can set his/her preferred colour settings in an iterative manner.

### Image Stabilization

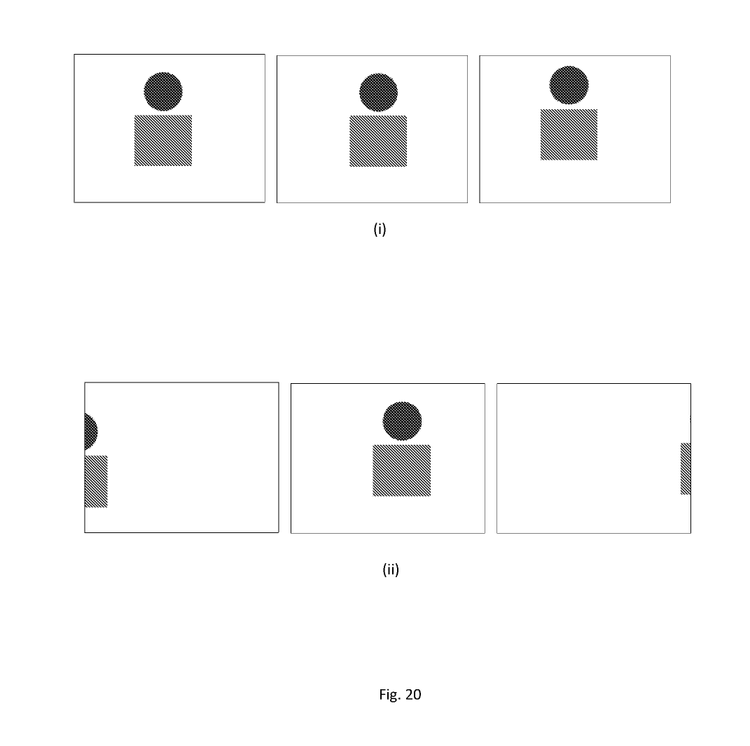

As shown in FIG. 6, system 400 is configured such that it can perform an image stabilization operation on the input video images. This helps users requiring image stability in their tasks. In general, this operation tracks global motion and depending on the scale of movement, the operation determines whether the user is stationary. If the user is stationary, motion compensation is performed on the input images to stabilize the images. By “stationary”, it is meant that the user is looking at a scene in which consecutive video image frames are roughly similar. There may be slight global motion but essentially most of the scene is the same. FIGS. 20(i) and (ii) respectively show consecutive video image frames as seen by a stationary user and a non-stationary user.

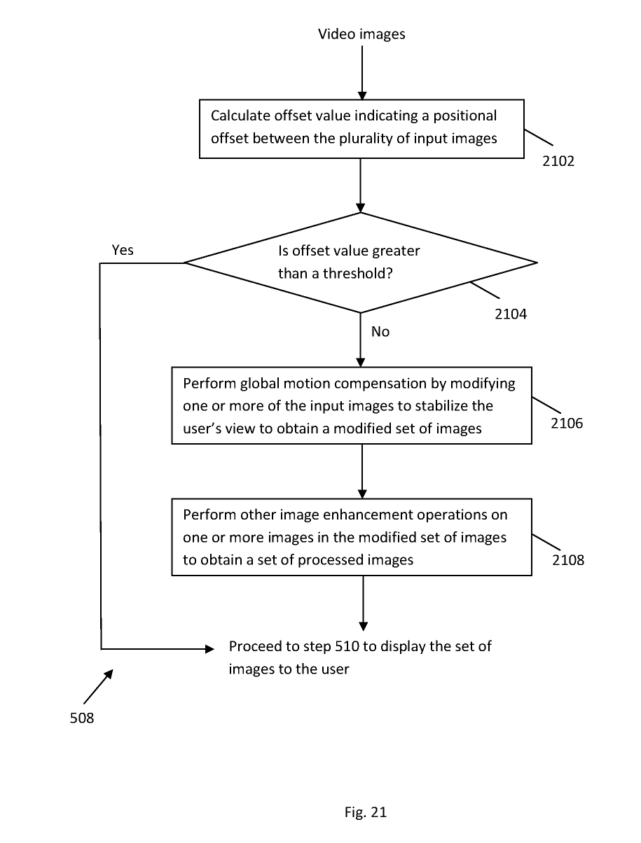

FIG. 21 shows how the image stabilization operation is performed in system 400. In particular, this image stabilization operation is performed via sub-steps 2102-2108 which are sub-steps of step 508 of method 500. The input to the image stabilization operation is a plurality of video images at proximate times (e.g. two or more successive images) and may either be most recently captured by the video camera 402 or been processed via one or more of the image enhancement operations shown in FIG. 6.

In sub-step 2102, global estimation is performed. In particular, an offset value indicating a positional offset between the plurality of input images is first calculated. The offset value may be calculated over all of the input images or only a subset of the input images.

In sub-step 2104, global motion classification is performed. More specifically, in sub-step 2104, the offset value is compared against a threshold. If the offset value is more than the threshold, it is determined that the user is not stationary (i.e. moving). In this case, the scene that the user is looking at is constantly changing so it is not necessary to perform image stabilization. Therefore, if the offset value is more than the threshold, the image stabilization operation ends and the images are displayed to the user. Else, it is determined that the user is stationary and sub-steps 2106-2108 are performed.

In sub-step 2106, global motion compensation is performed by making use of the portion of the scene that is similar as an anchor and stabilize the user view accordingly. In particular, one or more of the input images are modified based on the positional offset (as indicated by the offset value) to obtain a modified set of images.

In one example, the global estimation may be performed by first extracting images having a high degree of similarity from the plurality of input images, seeking respective anchor regions (for instance, regions of a predetermined size) in these extracted images and calculating the offset value as the positional offset between the anchor regions (for instance, the distance from the centre of one anchor region in one of the extracted images to the centre of another anchor region in another of the extracted images). In this example, the global motion compensation may be performed by using the offset value to modify one or more of the images to bring the anchor regions into alignment. This may be done by modifying successive ones of the input images to reduce the offset between the images.

In step 2108, other image enhancement operations (shown in FIG. 6) as desired by the user are performed on one or more images in the modified set of images to obtain a set of processed images. The set of processed images are then displayed to the user.

Various modifications will be apparent to those skilled in the art.

For example, although the distortion correction and the eccentric fixation operations are two important operations of system 400, it is not essential that the system 400 includes both of these operations. Rather, the system 400 can include any one or more of the image enhancement operations shown in FIG. 6 (i.e. eccentric fixation, distortion correction, vision enhancement, magnification and image stabilization) and optionally, other enhancement operations/processes.

Different ones of the image enhancement operations may be useful for different users. Distortion correction is usually needed before the visual field is damaged. Once there is permanent visual field damage, eccentric fixation is probably the only means by which the user can see properly. Although not essential as mentioned above, it is preferable for the system 400 to address both early and late visual field damage cases in order that the system 400 can be useful for a larger group of low vision users. Therefore, it is preferable to include both the distortion correction and eccentric fixation operations in system 400.

Further, system 400 need not contain a microphone for capturing voice commands. The user's commands may be in a different format and system 400 may instead or further comprise an alternative user input device. For example, system 400 may contain a keypad for the user to type his/her commands for processing by the system 400.

Method 500 need not comprise a step of generating the data characterizing the visual impairment. Instead, such data may be input by the user to system 400 for storage in the data storage device 406. The user also need not provide voice commands and in this case, the processing of step 508 may be performed based on the data characterizing the visual impairment alone.

After capturing an image using the video camera 402, the image may be processed using only one of the operations of system 400 as shown in FIG. 6 or using more than one of these operations. When more than one operation is performed on the image, the operations may be performed consecutively on the image before the image is displayed to the user, and the order of the operations may be varied according to the needs of the user. In other words, depending on the user requirements, sub-steps 708, 904, 1110, 1204, 1412, 1709, 1810, 2108 may comprise one or more of the operations shown in FIG. 6 or may be totally omitted. To ensure accurate calibration results, the operations performed in sub-step 708 are the same as those performed in sub-step 904. Similarly, the operations performed in sub-steps 1110 and 1709 are respectively the same as those performed in sub-steps 1204 and 1810.

In addition, although FIGS. 9, 12, 18, 21 show the sub-steps of the operations being performed only once, these sub-steps can be performed iteratively until the user is satisfied with the processed image. For example, for the distortion correction operation shown in FIG. 12, after performing sub-step 1202, the deformed image can be displayed to the user and if the user believes that more correction is required, the user can input a command to system 400 to repeat sub-step 1202, after which the further deformed image is displayed to the user for his/her review again. This can be repeated until the user is satisfied with the deformed image or until further deformation of the image makes little or no improvement to the image (this can be determined by asking the user if he/she sees a difference in the previously deformed image and the currently deformed image, or by calculating a difference in the pixel intensity values between the previously deformed image and the currently deformed image, and comparing this difference to a threshold to determine if the iteration should continue). For such an iterative operation, the overall correction matrix C can be expressed as C=C₁C₂ . . . Cₙ, where Cᵢ is the correction matrix calculated and applied in the i^{th} iteration, and the final deformed image is equivalent to applying the overall correction matrix C to the initial image (i.e. the image before any deformation).

The embodiments of the present invention have several advantages, some of which are described below.

The system 400 performs real time video image processing and can provide an augmented reality environment to assist and guide patients with low vision. In particular, the system 400 is able to capture real time video images (real time visual targets) as seen from a user's perspective (by objective and subjective ocular and visual tests) and can automatically enhance these images based on the user's needs (which are determined via calibration processes involving the patient's feedback).

Further, the system 400 can display augmented-reality images on transparent LCDs. More specifically, the image display device 410 can be configured to simultaneously display a plurality of overlying layers, one of the layers comprising the processed image and another one of the layers comprising the captured images. In this manner, the user can see the actual object of interest and the augmented layers (comprising the processed images) simultaneously. The system 400 can appear like a normal pair of spectacles, but with graphics including for example, augmented corrected zones, magnified and enhanced real time images, Amsler grid and markers corresponding to PRLs appearing within the user's visual field. This allows patients having visual problems to be able to view objects in the same manner as people with normal vision. The system 400 hence allows automated/semi-automated visual target tracking.

Moreover, the system 400 can provide patients automatic assistance in using their PRL and a customized field of view based on their needs by automatically characterizing each individual's visual abnormality using objective and subjective assessment techniques. Using the characterization outcome, the system 400 enhances images captured by the video camera to meet the patient's needs. In particular, the system 400 can determine a suitable PRL for the patient and guide the patient to use the PRL to view the images. Therefore, the system 400 can help enhance the vision of patients having eccentric fixation issues.

The system 400 may be used with the assistance of a visual therapist for fine-tuning purposes depending on the visual tasks required. However, the system 400 may also be used in the absence of an optometrist, hence helping to reduce the workload of optometrists. The system 400 is thus a useful rehabilitation tool for AMD patients with low vision.

The system 400 can also be integrated into a wearable product or a handheld tool, facilitating the use of it by the patient. The patient also can simply input voice commands to operate system 400. Thus, the system 400 can be easily integrated into a patient's daily life.

Unlike prior art low vision aids, the system 400 allows adjustment of the magnification of the images as per the patient's needs (based on commands input by the patient). The system 400 is also able to automatically correct distortions and enhance a patient's vision.

Table 1 below shows a comparison between the system 400 and prior art low vision aid devices. From Table 1, it can be seen that system 400 provides several advantages over prior art low vision aids, thereby helping to overcome the limitations of the prior art low vision aid devices.

TABLE 1

| | | Spectacle- | | | |
|---|---|---|---|---|---|
| | | mounted | | Intraocular | |
| | | aspheric | Portable | lens (IOP- | |
| | | hyperocular | video | Yip by | System 400 |
| | Amsler Grid | low vision aid | magnifiers | Soleko) | (ALVA) |
| Form | Can be in the | Attach onto | Handheld | Implantation | Wearable/ |
| | form of paper | glasses | device, not | of lens is | handheld tool |
| | or electronic | | modifiable | required | |
| | version | | | | |
| Guidance | Requires | — | — | Implantation | Augmented- |
| needed? | optometrist's | | | to be done by | reality |
| | guidance | | | experienced | guidance |
| | | | | surgeons | |
| Magnification | — | Fixed | Adjustable | Fixed | Adjustable |
| | | magnification | magnification | magnification | magnification |
| | | level | level | level | level |
| How to | — | — | Buttons on the | — | Voice |
| operate? | | | device | | commands |
| Any image | No | No | Yes | No | Yes |
| enhancement | | | | | |

1-28. (canceled)

29. A system for displaying a video image to a user having a visual impairment, the system comprising:
a video camera for capturing images;
a data storage device for storing data characterizing the visual impairment;
a processor for processing the captured images in real time in dependence on the data, to generate processed images; and
an image display device for displaying the processed images in real time in a display region for viewing by the user;
wherein the processor is operative to perform at least one of the operations of:
(i) including in the display region a marker at a location determined by said data offset from a centre of the display region, whereby if the user, who suffers from blurred vision at the centre of the user's visual field, looks at the marker, the user sees the captured image in focus; or
(ii) deforming a portion of the captured image which depends on the data, by a correction matrix which depends upon the data, whereby, upon displaying the processed images by the display device, the correction matrix corrects a deformation in a corresponding portion of the visual field of the user due to the visual impairment.

30. A system according to claim 29, wherein the processing of the image includes translating the image to the location of the marker in the display region.

31. A system according to claim 29, further comprising a microphone for capturing a voice command spoken by the user, the processor being operative to recognize the command and modify the real time processing of the captured images in dependence on the recognized command.

32. A system according to claim 29 which is adapted for wearing by a user by further comprising elements operative to attach the system to the user.

33. A system according to claim 29, wherein the image display device is configured to simultaneously display a plurality of overlying layers, one of the layers comprising the processed images and another one of the layers comprising the captured images.

34. A method for displaying a video image to a user having a visual impairment, the method comprising:
capturing images with a video camera;
processing the captured images in real time in dependence on data characterizing the visual impairment, to generate processed images; and
displaying the processed images to the user in real time in a display region;
wherein the processing includes at least one of the operations of:
(i) including in the display region a marker at a location determined by said data offset from a centre of the display region, whereby when the processed images are displayed to the user, who suffers from blurred vision at the centre of the user's visual field, and the user looks at the marker, the user sees the captured image in focus; or
(ii) deforming a portion of the captured image which depends on the data, by a correction matrix which depends upon the data, whereby the correction matrix corrects a deformation in a corresponding portion of the visual field of the user due to the visual impairment.

35. A method according to claim 34, wherein the processing of the image includes translating the image to the location of the marker in the display region.

36. A method according to claim 34, wherein the processing of the image includes a magnification of the image by a selectable magnification factor.

37. A method according to claim 36, wherein the processing of the image further includes identifying an object of interest in the image and adjusting a location of the image in the display region based on the identification after the magnification of the image.

38. A method according to claim 34, wherein the processing further includes an adjustment of any one or more of the colour, the contrast or the sharpness of the captured images.

39. A method according to claim 38, wherein the processing further includes for each of the captured images, determining if the image comprises text and only performing the adjustment of any one or more of the colour, the contrast or the sharpness of the image if the image comprises text.

40. A method according to claim 38, wherein the processing includes changing one or both of foreground and background colours of the image.

41. A method according to claim 34, wherein the processing includes modifying successive ones of the captured images to reduce an offset between the images.

42. A method according to claim 41, further including measuring an offset value between successive captured images, and determining whether the offset value is below a threshold, and only performing the modification if the offset value is below a threshold.

43. A method according to claim 41, wherein modifying successive ones of the captured images comprises identifying anchor regions in the images and modifying the images to bring the anchor regions into alignment.

44. A method according to claim 34, further comprising:
capturing a voice command spoken by the user,
recognizing the command, and
modifying the real time processing of the captured images in dependence on the recognized command.

45. A method according to claim 34, further including a step, performed prior to said capturing, of generating the data by measuring characteristics of the visual impairment.

46. A method according to claim 45, wherein the correction matrix is generated by obtaining a user distortion matrix characterizing an image deformation caused to at least a portion of the visual field of the user by the visual impairment, and inverting the user distortion matrix.

47. A method according to claim 46, wherein the user distortion matrix is obtained based on a portion of the image seen as deformed by the user.

48. A method according to claim 47, wherein the user distortion matrix comprises intensity values of pixels of the portion of the image seen as deformed by the user.
